(19) United States
(12) Patent Application Publication
(10) Pub. No.: US 2015/0219544 A1
(43) Pub. Date: Aug. 6, 2015

(54) CELL OR PARTICLE ANALYZER AND SORTER

(71) Applicant: Microsensor Labs, LLC., Chicago, IL (US)

(72) Inventor: Peng Liu, Chicago, IL (US)

(73) Assignee: Microsensor Labs, LLC., Chicago, IL (US)

(21) Appl. No.: 14/171,661

(22) Filed: Feb. 3, 2014

(51) Int. Cl. G01N 15/10; G01N 33/574

(52) U.S. Cl. CPC G01N 15/1031; G01N 33/57492; G01N 2015/1081; G01N 2015/1006; G01N 2015/0065

(57) ABSTRACT

A cell analyzing and sorting apparatus includes a substrate, a detection device embedded in a substrate configured to detect cells labeled with magnetic beads, a focusing device embedded in the substrate configured to align the labeled cells with and introduce the labeled cells serially to the detection device, and a sorting device in the substrate configured to separate the labeled target cells passing through the detection device. The detection device comprises a plurality of magnetic sensing elements. Each of the magnetic sensing elements comprises a Hall-effect sensor configured to detect a magnetic characteristic of a magnetic bead labeled to a cell.

### TECHNICAL FIELD

Embodiments of this disclosure relate to methods and apparatuses for detecting analytes in a biological sample. In particular, methods and apparatuses for detecting target analytes using magnetic beads and Hall-effect sensors are described.

### BACKGROUND

In both clinical applications and basic science, efficient isolation of rare cells from biological samples is critical. One example is Circulating Tumor Cells (CTCs). Most cancer-related deaths are caused by metastasis, the dissemination of cancer cells from the primary tumor through the bloodstream to new organ sites. Compared to fresh tissue biopsy, “liquid biopsy” of CTCs in blood samples is much more accessible, affordable and convenient, and is much less invasive. Some studies indicate that the systematic dissemination of rare cancer cells can occur early during cancer progression. Other clinical studies show that CTC counts can be used as prognostic indicators of survival for a variety of cancers. In addition, longitudinal analysis of CTCs can provide information on treatment responses. Moreover, isolating CTCs for molecular analysis in order to genotype patient-specific tumors could eventually guide the targeted therapy based on genetic changes. For all these reasons, the detection, enumeration and isolation of CTCs are critical to early cancer detection, personalized therapy and treatment monitoring.

Despite their significant clinical relevance, research on CTCs is hampered by lack of affordable and automated tools that can efficiently isolate them from biological samples. The conventional technique for analyzing and sorting single cells is fluorescence-activated cell sorting (FACS), a flow cytometry method that combines optical detection with electrostatic deflection. However, FACS is very inefficient with rare events, and CTCs in a patient's blood sample can be as low as 1 CTC in a milliliter containing 10⁹ normal blood cells. In these cases, FACS takes extremely long processing times and suffers from significant losses in yield and purity. In addition, since FACS uses a droplet-based sorter, its instruments suffer from cell contamination and potential biohazards when processing live cells and infectious agents. Further, fluorescence-activated cell sorting devices are generally more expensive.

The ideal isolation method should be highly sensitive, reproducible and easy to implement in a clinical setting. Due to the low concentration of target cells in the bloodstream, a first enrichment step must often be carried out to increase the sensitivity of the assay. This is followed by a detection/sorting step that will ideally protect the integrity of detected CTC, allowing additional biological characterization.

Currently there is no efficient tool for rare cell detection/sorting that bridge the gap between sample enrichment and downstream molecular analysis. The most widely used CTC enrichment approaches rely on antibodies against the epithelial cell adhesion molecule (EpCAM), a surface marker that is expressed in CTCs but absent from normal leukocytes. The most notable technique for isolating the cells is magnetic-activated cell sorting (MACS) where potential target cells are immunomagnetically selected with magnetic beads. However, although MACS can enrich a sample by as much as 10⁵ fold, the sample usually contains leukocyte contamination of over 9,000/ml, causing purities as low as 0.02% in samples with a low occurrence of CTCs. Therefore downstream molecular characterization still requires single cell analysis and sorting that identifies target cells and purifies samples.

Another problem lies in most CTC analysis techniques being based on fluorescent labels. For example, in conventional methods, the immunomagnetically enriched sample is stained and then scanned with fluorescence microscopy to identify and enumerate CTCs. This approach requires extra sample processing steps (e.g., pre-fixation), expensive optical instruments and complex equipment setups. Moreover, for use in downstream analysis, these identified cells must still be isolated manually or semi-automatically, a very labor-intensive and time-consuming procedure. This extensive amount of sample processing and the lack of full automation often cause cell loss and contamination, limiting the possibility for further biological characterization.

Accordingly, there is a general need for an improved method and apparatus that can overcome the above prior art problems. There is a need for a method and apparatus for single cell analysis, enumeration, and sorting that can be seamlessly integrated with immunomagnetic enrichment techniques and microfluidics to isolate rare cells with high purity and viability, and thus allows further molecular analysis.

### SUMMARY

In some embodiments, a cell analyzer and sorter is provided to recover rare cells from whole blood with high purity and viability. Highly purified and viable cells are essential for downstream molecular analysis to characterize mutational heterogeneity that can provide significant implications ranging from cancer prognostics to stem cell therapy. Currently, research on rare cells is hampered by the lack of efficient and automated tools for cell analysis and sorting due to their extraordinarily rare occurrence. For such rare events, conventional fluorescent-activated cell sorting (FACS) techniques suffer from extremely long processing time and significant loss in yield and purity.

In some embodiments, a magnetic flow cytometer for rare cell analysis, enumeration and sorting is provided, which can be seamlessly integrated with high-throughput immunomagnetic enrichment techniques such as magnetic-activated cell sorting (MACS) and microfluidic systems to recover rare cells with high purity and viability. Individual magnetic bead-labeled cells are aligned in a microfluidic channel by magnetic forces and serially passed onto a magnetic detector for analysis and profiling of surface markers. Each cell is then magnetically manipulated to either target or waste channel depending on the detector output. All the functions of the magnetic flow cytometer are based on the properties of magnetic bead labels. By using magnetic bead labels instead of fluorescent labels, the disclosed apparatuses and methods can not only eliminate the need for bulky and expensive optical components but also significantly simplify sample preparation.

Various other embodiments are also described herein, including low-cost microelectronic/microfluidic devices that automate the functions of multiplexed identification and separation of rare cells, a single cell surface marker analysis method by simultaneous detection of various magnetic bead labels based on their Néel relaxation signatures, and methods for effective recovery of rare cells with high purity and viability from the whole blood. The disclosed methods and apparatuses significantly advance the current technology in rare cell analysis, and improve cellular diagnostics and personalized therapy.

This Summary is provided to introduce selected embodiments in a simplified form and is not intended to identify key features or essential characteristics of the claimed subject matter, nor is it intended to be used as an aid in determining the scope of the claimed subject matter. Other embodiments of the disclosure are further described in the Detail Description.

### BRIEF DESCRIPTION OF THE DRAWINGS

These and other features and advantages of the disclosed methods and apparatuses will become better understood upon reading of the following detailed description in conjunction with the accompanying drawings and the appended claims provided below, where:

FIG. 1 is a flowchart illustrating the steps of an exemplary method according to some embodiments of this disclosure;

FIGS. 2A-2C illustrate the structure and magnetic property of an exemplary magnetic microbead;

FIG. 3 illustrates the contents in an enriched sample according to some embodiments of this disclosure;

FIG. 4 is a schematic diagram showing functional blocks of an exemplary apparatus according to some embodiments of this disclosure;

FIGS. 5A-5D show an exemplary detection device fabricated in a CMOS chip according to some embodiments of the disclosure;

FIG. 6 schematically shows the working principle of a Hall-effect sensor;

FIGS. 7A-7C compare a conventional magnetic bead detection method with an exemplary method according to some embodiments of the disclosure;

FIG. 8 shows magnetic responses of three types of beads with characteristic relaxation signatures;

FIG. 9A illustrates an optical image under microscope showing a target cell and various contaminants on a detector array;

FIG. 9B illustrates a magnetic detector output showing detector responses to various particles;

FIG. 10 schematically shows magnetic focusing and sorting according to some embodiments of this disclosure; and

FIGS. 11A-11B are block diagrams showing a system including a magnetic flow cytometer chip interfacing with a computer.

### DETAILED DESCRIPTION

Various embodiments of methods and apparatus for detecting analytes in a sample are described. It is to be understood that the disclosure is not limited to the particular embodiments described as such may, of course, vary. An aspect described in conjunction with a particular embodiment is not necessarily limited to that embodiment and can be practiced in any other embodiments. For example, while various embodiments are described in conjunction with rare cancer cells such as circulating tumor cells (CTCs) in a blood sample for illustrative purpose, the claimed invention can be practiced to detect various other analytes in any other samples. Further, in the following description, numerous specific details such as examples of specific components, dimensions, processes, etc. may be set forth in order to provide a thorough understanding of the disclosure. It will be apparent, however, to one of ordinary skill in the art that these specific details need not be employed to practice embodiments of the disclosure. In other instances, well known components or steps may not be described in detail in order to avoid unnecessarily obscuring the embodiments of the disclosure.

As used in the description and appended claims, the singular forms of “a,” “an,” and “the” include plural references unless the context clearly dictates otherwise. Thus, for example, reference to “a target analyte” includes one or more target analytes, and reference to “the magnetic bead” includes one or more magnetic beads of the characteristics described herein. The terms “first” and “second” may be used herein to distinguish one element from another element in describing various elements e.g. two or more than two elements. The use of the terms “first” and “second” should not be construed as that the embodiment is limited literally to two elements. All technical and scientific terms used herein have the same meaning as commonly understood by one of ordinary skill in the art to which this invention belongs, unless defined otherwise.

As used herein, the term “magnetic bead” refers to a microbead comprising magnetic nanoparticles dispersed in a polymer or silica matrix. The magnetic beads used in this disclosure may be superparamagnetic, i.e., only magnetic when a magnetizing field is applied.

As used herein, the term “target analyte” refers to an analyte of interest that can be detected by the method and apparatus of this disclosure. A target analyte may be a cancer cell such as a circulating tumor cell in a blood sample and any other analytes of interest in a sample fluid.

As used herein, the term “contaminant” refers to any non-target substance or particle in a sample that contains target analytes. For example, a contaminant may be a free magnetic bead, a non-target cell such as leukocyte, and dust, etc.

A method of detecting target analytes is described in this disclosure. In some embodiments, a mixture comprising target analytes and contaminants is provided. The target analytes may be labeled with at least first magnetic beads and the contaminants may be labeled with second magnetic beads, wherein the first magnetic beads have a first magnetic relaxation characteristic and the second magnetic beads have a second magnetic relaxation characteristic. The mixture comprising the labeled target analytes and contaminants may be introduced to a detection region to allow a labeled target analyte or a labeled contaminant passing through the detection region one at a time. A magnetic field is applied to the detection region, thereby magnetizing the first magnetic beads in the labeled target analyte or the second magnetic beads in the labeled contaminant in the detection region. The magnetic field is then removed. The magnetic relaxation characteristics of the magnetized first magnetic beads or of the magnetized second magnetic beads are detected after the magnetic field is removed. A target analyte or a contaminant can be differentiated based on the detected magnetic relaxation characteristics.

The target analytes may be cells expressing a first surface marker. The contaminants may be non-target cells expressing a second surface marker. The first magnetic beads may be coated with a first antibody specific to the first surface marker, and the second magnetic beads may be coated with a second antibody specific to the second surface marker. By way of example, the target analytes may be circulating cancer cells (CTCs) expressing EpCAM and the contaminants may be leukocytes expressing CD45. The first magnetic beads may be coated with an anti-EpCAM antibody and the second magnetic beads may be coated with an anti-CD45 antibody. Alternatively, the target analytes may be circulating cancer cells (CTCs) expressing HER2, the contaminants may be leukocytes expressing CD45, and the first magnetic beads may be coated with an anti-HER2 antibody and the second magnetic beads may be coated with an anti-CD45 antibody.

In some embodiments, the target analytes may be labeled with two or more types of magnetic beads, e.g., first magnetic beads and third magnetic beads. As such, the first and third magnetic beads in a labeled target analyte or the second magnetic beads in a labeled contaminant can be magnetized by the magnetic field. The magnetic relaxation characteristics of the magnetized first and third magnetic beads or of the magnetized second magnetic beads can be detected after the magnetic field is removed. The target analyte and contaminant can be differentiated based on the detected magnetic relaxation characteristics of the magnetized first and third magnetic beads and of the magnetized second magnetic beads.

The target analytes may be cells expressing a first surface marker and a third surface marker, and the contaminants may be non-target cells expressing a second surface marker. The first magnetic beads may be coated with a first antibody specific to the first surface marker, the third magnetic beads may be coated with a third antibody specific to the third surface marker, and the second magnetic beads may be coated with a second antibody specific to the second surface marker. For example, the target analytes may be CTCs expressing EpCAM and HER2, the contaminants may be leukocytes expressing CD45. The first magnetic beads may be coated with anti-EpCAM antibody, the third magnetic beads may be coated with anti-HER2 antibody; and the second magnetic beads may be coated with anti-CD45 antibody. One of the purposes of using two types of beads coated with two types of antibodies is to obtain a profile of surface markers of the target cells for flow cytometer analysis.

The magnetic relaxation characteristics of the magnetized first or second or third magnetic beads may be detected by CMOS Hall-effect sensors. The magnetic relaxation characteristics can be Néel relaxation time constant of the magnetized first or second magnetic beads after the magnetic field is removed.

The labeled target analytes and labeled contaminants in the mixture may be caused to pass to the detection region by a magnetic force. After the detection, the labeled target analyte and contaminant can be sorted. The labeled target analyte and contaminant may be sorted and directed to designated paths using a magnetic force.

In a specific embodiment, a method of detecting target cells labeled with multiple types of magnetic beads is described. In the method, the target cells in a sample may be labeled with first magnetic beads and second magnetic beads, wherein the first magnetic beads have a first magnetic relaxation characteristic and the second magnetic beads have a second magnetic relaxation characteristic. A magnetic field may be applied to a labeled target cell, thereby magnetizing the first and second magnetic beads in the labeled target cell. The applied magnetic field is then removed, and the magnetic relaxation characteristics of the magnetized first and second magnetic beads are detected. The target cell can be determined based on the magnetic relaxation characteristics of magnetized first and second magnetic beads.

The target cells may expresses a first surface marker and a second surface marker, and the first magnetic beads may be coated with a first antibody specific to the first surface marker and the second magnetic beads may be coated with a second antibody specific to the first surface marker. For example, the target cells may be circulating cancer cells (CTCs) expressing the first surface marker of EpCAM and the second surface marker of HER2, and the first magnetic beads may be coated with anti-EpCAM antibody and the second magnetic beads may be coated with anti-HER2 antibody.

The magnetic relaxation characteristics may include Néel relaxation time constant of the magnetized first and second magnetic beads. The magnetic relaxation characteristics may be detected by CMOS Hall-effect sensors or other magnetic sensors.

A cell analyzing and sorting apparatus is described in this disclosure. The apparatus may include a substrate, a detection device embedded in the substrate configured to detect cells labeled with magnetic beads, and a focusing device embedded in the substrate configured to align the labeled cells with and introduce the labeled cells serially to the detection device. The detection device may include a plurality of magnetic sensing elements. Each of the magnetic sensing elements may include a Hall-effect sensor configured to detect a magnetic characteristic of a magnetic bead labeled to a cell.

The plurality of magnetic sensing elements may be arranged in arrays of rows and columns. Each magnetic sensing element may have a size about same as a size of a magnetic bead labeled to a cell. For example, each magnetic sensing element may have a size about 5×5 micrometers. By way of example, each magnetic sensing element may include a N-well plate and a pair of metal wires each being disposed adjacent along a side of the N-well plate configured to generate a magnetic field for magnetizing a magnetic bead labeled to a cell.

The focusing device may include one or more current conducting wires configured to generate a magnetic force for moving the labeled cells to the detection device.

The apparatus may further include a sorting device embedded in the substrate configured to isolate target cells from contaminants passing through the detection device. The sorting device may include a plurality of current conducting wires configured to generate magnetic fields for directing target cells and contaminates in designated paths.

The focusing device, the detection device, and the sorting device may be fabricated in a single CMOS chip.

The apparatus may include a controller configured to control the operation of the detection device, the focusing device, and the sorting device. The apparatus may also include a computer display configured to display the output of the plurality of magnetic sensing elements.

In a specific embodiment, an integrated apparatus includes a substrate, a detection device embedded in the substrate and configured to detect cells labeled with magnetic beads, a focusing device embedded in the substrate and configured to align the labeled cells with and introduce the labeled cells serially to the detection device, and a sorting device embedded in the substrate and configured to isolate target cells from contaminants passing through the detection device. The detection device may include a plurality of magnetic sensing elements each comprising a Hall-effect sensor configured to detect a magnetic characteristic of a magnetic bead labeled to a cell. The integrated apparatus may be fabricated in a single CMOS chip.

The integrated apparatus may include a controller configured to control the operations of the focusing device, the detection device, and the sorting device. The controller may be configured to receive output from the plurality of magnetic sensing elements and provide signals to the sorting device for isolating target cells from contaminants based on the output. The apparatus may further include a computer display configured to display the output of the plurality of magnetic sensing elements.

Exemplary embodiments will now be described with reference to the figures. It should be noted that some figures are not necessarily drawn to scale and some well known components may not be shown. The figures are only intended to facilitate the description of specific embodiments, and are not intended as an exhaustive description or as a limitation on the scope of the disclosure.

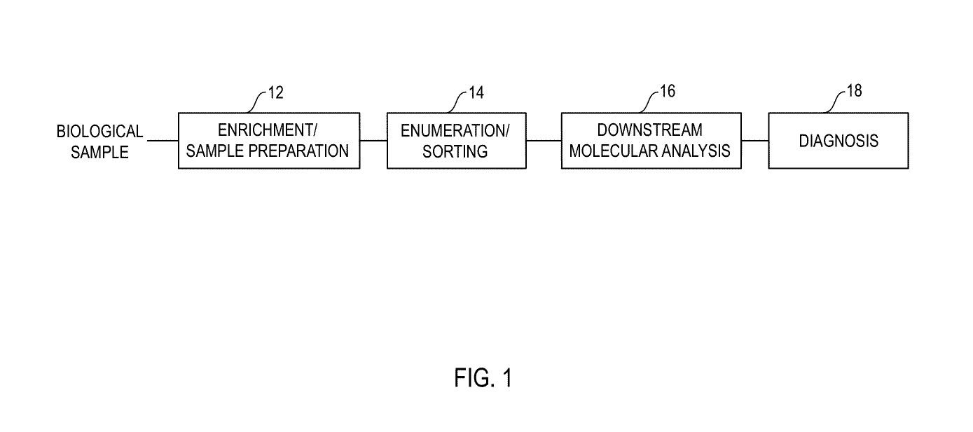

FIG. 1 is a flowchart illustrating the steps of an exemplary method according to some embodiments of this disclosure. In general, the method may comprise a sample preparation or enrichment step (step 12) and a detection step (step 14), which may in turn include analyte enumeration and sorting as will be described in greater detail below. The method may further include the steps of downstream molecular analysis (step 16) and diagnosis (step 18).

Sample preparation or enrichment may be conducted in situations where the presence of target analytes in a sample is rare. For example, efficient isolation of circulating tumor cells (CTCs) with high viability and purity is critical for cell culture and downstream molecular assays, but is technically challenging due to the rare occurrence of CTCs. The CTCs are 1-100 per milliliter in patient blood, mixed with over 1 million leukocytes (white blood cells) and 1 billion erythrocytes (red blood cells) per milliliter. The purity of CTCs in a biological sample is about 10⁻⁹. The term “purity” refers to the ratio of target cell count over total cell count in a sample. After a sample preparation or enrichment step, the sample purity may reach about 10⁻⁴, which still may not meet the requirement for cell culture and downstream assays. Through the method described in this disclosure, the sample purity can be further increased by more than 1000 times.

The starting sample may be prepared or enriched using immunomagnetic enrichment techniques known in the art. For example, in a sample containing target analytes and contaminants, a first type of magnetic beads coated with an antibody specific to a surface marker expressed by the target analyte may be added to capture the target analytes. After a period of incubation, the sample may be placed on a magnet to isolate the bead-bound target analytes. The isolated sample may still contain a large number of contaminants. A second type of magnetic beads coated with an antibody specific to a surface marker expressed by the contaminant cells may be added to capture the contaminant cells. The enriched sample can then be introduced to the apparatus as will be described in detail below for enumeration and sorting. Optionally, the enriched sample may be further processed by filtration to remove free beads and small particles.

By way of example, an exemplary sample may include cells from a human breast carcinoma cell line MDA-MB-453 that expresses two epithelial surface markers, epithelial cell adhesion molecule (EpCAM) and/or human epidermal growth factor receptor 2 (HER2). In this specific example for illustrative purpose, about 1 to 500 MDA-MB-453 cells may be spiked into 7.5 ml of a healthy human volunteer's whole blood. A cocktail of anti-EpCAM or anti-HER2 antibodies bound to one type of magnetic beads (bead type 1) may be added to the sample to specifically capture epithelial cell after a short incubation. The bead-bound target cells may be isolated from whole blood when the tube containing the sample is placed on a magnet. The isolated sample may still contain a large amount of leucocytes and may be mixed with anti-CD45 coated magnetic beads (bead type 2) to label leukocytes. The sample may be further processed through a membrane filter of 10-μm pore size to remove most of the free beads and small particles (e.g. residual red blood cells). Other methods such as lysis or centrifuge may also be used to remove residual red blood cells.

Various commercially available magnetic beads can be used in the immunomagnetic labeling according to embodiments of this disclosure. Unlike their fluorescent counterparts, immunomagnetic bead labels are very stable over time and not susceptible to photobleaching. These beads are superparamagnetic, i.e., only magnetic when a magnetizing field is applied. Exemplary magnetic beads include Dynabeads®, commercially available from Thermo Fisher Scientific in Waltham, Mass.

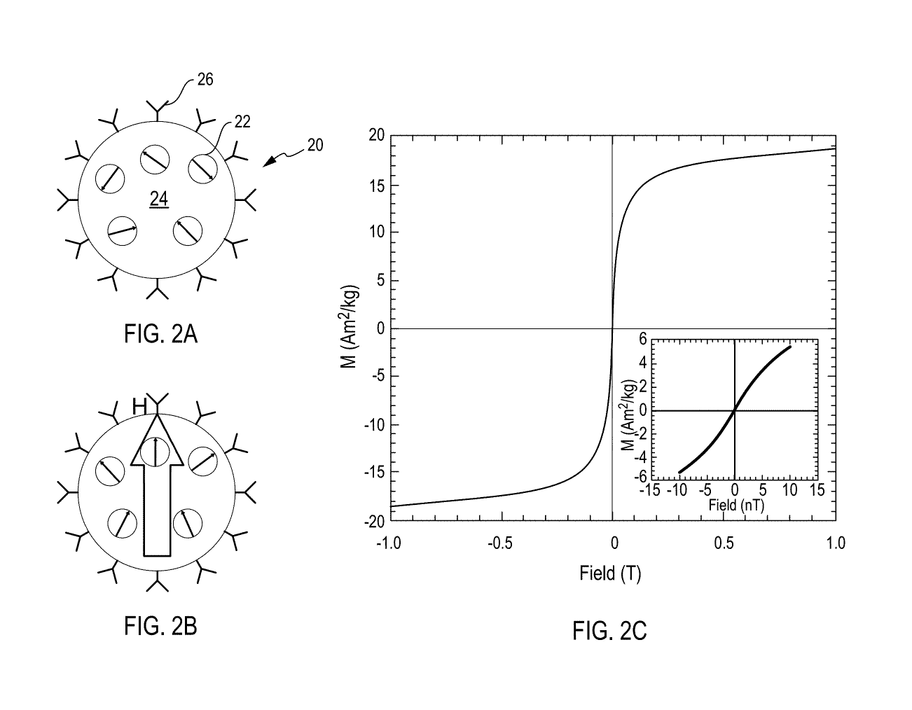

FIGS. 2A-2C show the structure and magnetic property of an exemplary magnetic microbead 20. The magnetic bead 20 may include magnetic nanoparticles (MNPs) 22 dispersed in a matrix 24 of polymers, silica or hydroxylapatite etc. The magnetic nanoparticle 22 may be magnetic elements such as iron, nickel and cobalt and their chemical compounds. By way of example, iron-oxide, including magnetite, Fe₃O₄, and its oxidized form maghemite, γ-Fe₂O₃ nanoparticles are commercially available and widely used. The size of magnetic nanoparticles 22 may range from several nanometers to several microns. The size of microbead 20 may range from tens of nanometers to tens of microns.

A magnetic nanoparticle 22 becomes superparamagnetic when it is composed of a single magnetic domain. Such a superparamagnetic nanoparticle exhibits its magnetic behavior only when an external magnetic field is applied. When the external magnetic field is switched off, its residual field falls to zero and thus prevents magnetic nanoparticles from agglomeration.

Each MNP 22 in the bead 20 may be considered a tiny magnet. When no external field is applied (H=0), the MNPs 22 are randomly oriented and thus, the bead 20 does not have a magnetic field, as shown in FIG. 2A. When an external field is applied (H>0), the magnets tend to align with the external H and as a result, the bead 20 becomes magnetized and has a magnetic moment, as shown in FIG. 2B. FIG. 2C is DC magnetization curve of an exemplary bead vs. the applied magnetizing field. The inset in FIG. 2C shows the bead magnetization is 0 when no external field is applied.

The magnetic bead 20 may be coated with various functional groups, biomolecules, or ligands 26 for various biomedical applications. For example, the magnetic beads 20 may be coated with an antibody, protein or antigen, DNA/RNA probe or any other molecule with an affinity for the desired target. In cell isolation for example, the magnetic particles 20 may be coated with antibodies that bind specifically to the antigens on the target cell surface.

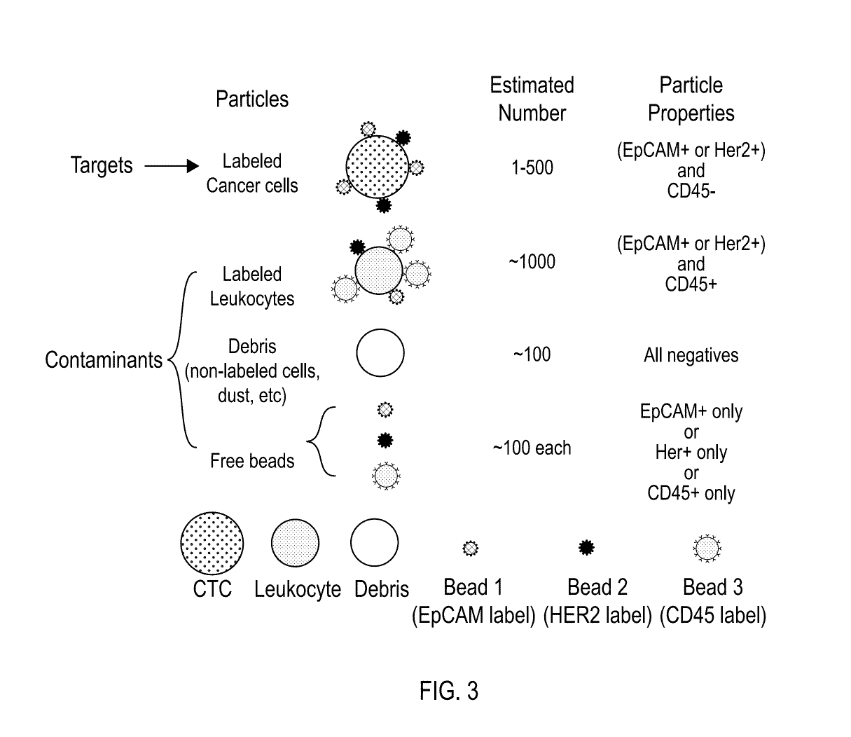

In a specific example, cancer cell line MDA-MB-453 that expresses two epithelial surface markers, EpCAM and/or HER2 was added to a human whole blood sample. A cocktail of anti-EpCAM and anti-HER2 antibodies bound to two types of magnetic beads were used to capture MDA-MB-453 cells added into human whole blood. The sample was prepared and enriched via commercial systems, such as Magnetic Activated Cell Sorting (MACS) or microfluidic systems. The enriched product containing beads-labeled cells and contaminants were further sorted in the present apparatus as will be described in more detail below. To accurately eliminate contaminating leukocytes, an additional mix of a third-type of beads conjugated with antibody against CD45 (leukocytes surface antigen) was added. The processed mixture would contain a small number of cancer cells and a large number of contaminants (mostly leucocytes, bead and debris), as shown in FIG. 3. FIG. 3 illustrates the contents of an enriched sample according to some embodiments of this disclosure. As shown, the enriched sample may include target cancer cells labeled with magnetic beads that are coated with EpCAM and HER2 antibodies. The enriched sample may also include a large number of contaminants, including labeled leukocytes, debris (non-labeled cells, dust, etc.), and free beads.

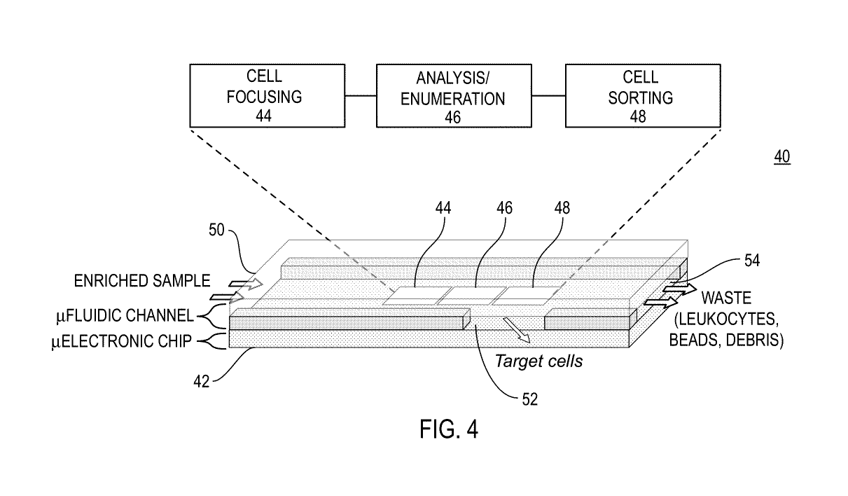

FIG. 4 is a schematic diagram showing functional blocks of an exemplary apparatus 40 according to some embodiments of this disclosure. The apparatus 40 shown in FIG. 4 can be used in detection/enumeration/sorting of target analytes in a sample according some embodiments of the disclosure (step 14 in FIG. 1). The apparatus 40 may include a substrate 42, a focusing device 44, a detection device 46, and a sorting device 48 embedded in the substrate 42. The apparatus 40 may also include a microfluidic channel 50 for introducing a sample, and a target channel 52 and a waste channel 54 for sorting target analytes and contaminates after passing through the detection device 46. In some embodiments, the focusing device 44, the detection device 46, and the sorting device 48 may be fabricated in a single CMOS chip.

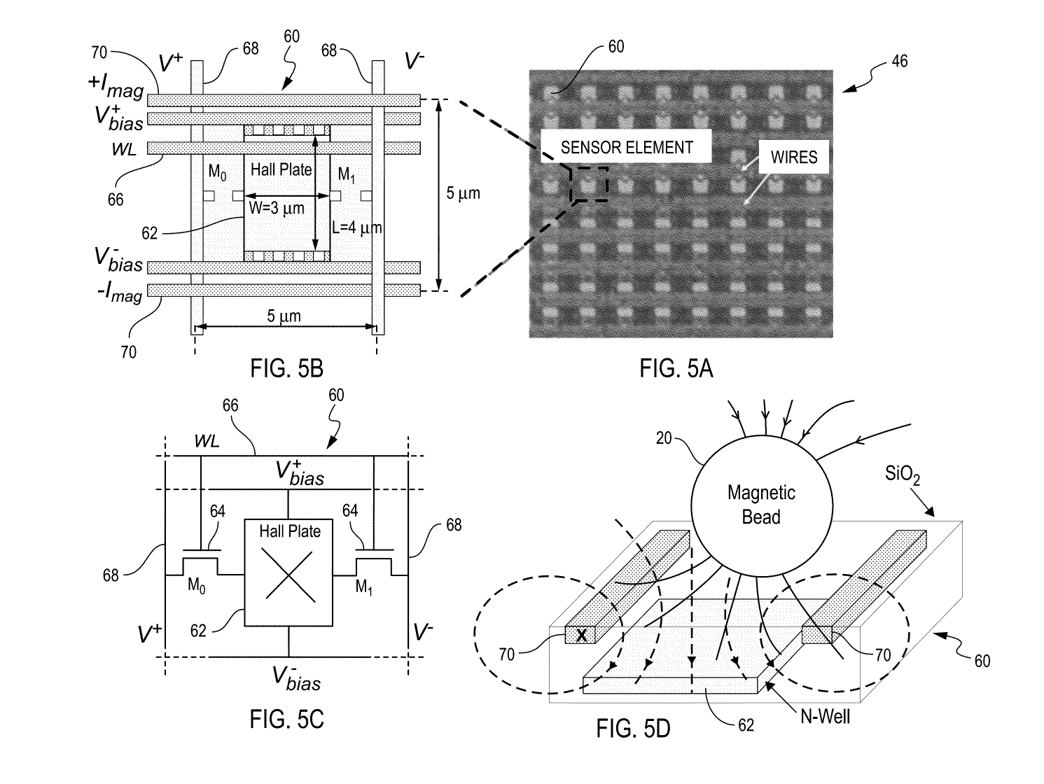

FIGS. 5A-5D show an exemplary detection device 46 fabricated in a CMOS chip according to some embodiments of the disclosure. FIG. 5A shows the detection device 46 including a plurality of magnetic sensing elements 60 arranged in an 8×8 array. FIG. 5B shows the layout of a single magnetic sensing element 60. FIG. 5C shows a schematic diagram for a single magnetic sensing element 60. FIG. 5D illustrates the principle of detecting a magnetic bead 20 on a CMOS Hall-effect sensor 60 (simplified in 3D). As shown in FIGS. 5A-5D, each magnetic sensing element 60 may include a Hall plate 62 and two access transistors 64 controlled by a word line (WL) 66. Each word line 66 may be shared by sensors in the same row. As such, the Hall sensor outputs (V⁺& V⁻) in each row can be read out in parallel. In each sensing element 60, a pair of current-carrying metal wires 70 (+I_{mag} & −I_{mag}) may be covered with silicon oxide and located about 1 μm from sensor surface 62. The pair of current-carrying metal wires 70 may generate a magnetizing field (dash lines in FIG. 5D) to magnetize the bead 20 or induce a magnetic field from the bead (solid lines). The induced magnetic field from the bead 20 may be detected by the embedded Hall plate 62 and converted to an electrical signal. The Hall-plate 62 may be implemented in the N-well layer of standard CMOS process. The standard CMOS process is well known in the art and its detail description is omitted herein for clarity of description of embodiments of this disclosure.

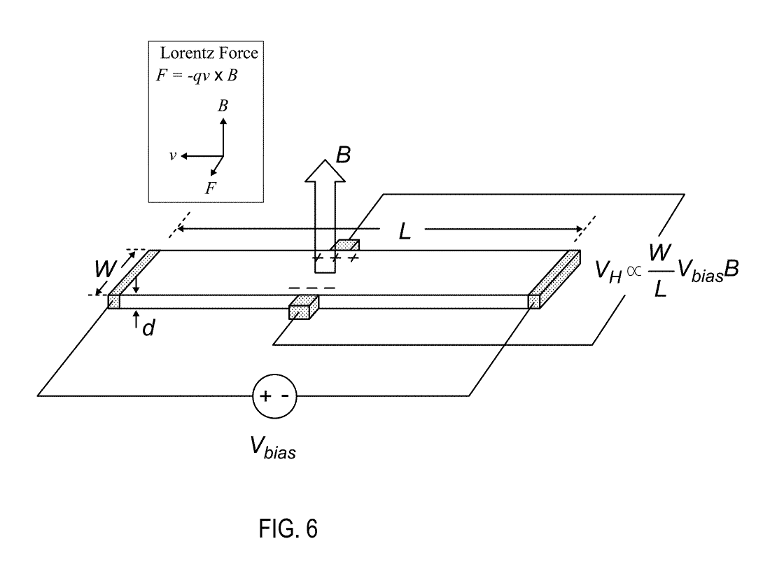

The operation and principle of a Hall-effect sensor is also well known and their detail description is omitted herein to avoid obscuring description of embodiments of this disclosure. Briefly, for a current-carrying conductor plate in a magnetic field transverse to the current direction, Lorentz force causes the charges to move along a curve path and therefore a Hall voltage that is proportional to the external magnetic field to develop across the plate, as shown in FIG. 6.

In conventional magnetic detection methods, the induced magnetic field from a bead is detected while the magnetizing field is on. However, detecting the magnetic field from a bead in the presence of a much larger magnetizing field imposes stringent requirements on the detector's dynamic range, offset, linearity, and temperature stability. For example, a commercially available M-280 Dynabead in a 10-mT external field generates a field less than 20 μT if measured 10-μm away from the bead center. This induced magnetic field from the bead is more than 50 dB lower than the magnetizing field (the “baseline”). Prior art techniques attempt to resolve a miniscule change from a bead superimposed on the much larger baseline. Since the baseline is sensitive to environmental variations, these solutions generally require reference sensors, baseline calibration and/or active temperature stabilization. These functional blocks, however, not only make the device less user-friendly, but also add significant penalty on chip area, power consumption and detection time.

According to some embodiments of this disclosure, the detection of the induced magnetic field from a bead starts after the magnetizing field is removed rapidly. The bead can be first magnetized by a large magnetizing field generated on-chip. Then, the magnetizing field is removed, and the decaying magnetic field from the bead or the magnetic relaxation characteristic or signature from the bead is detected.

The magnetic relaxation characteristic of a bead can be measured by the Néel relaxation. When the magnetizing field is turned off abruptly, the beads signal will decay to zero following its Néel relaxation time constant, described by the Néel-Brown model:

τ=τ₀e^{KV/kT} (I)

where τₒ is material dependent and usually around 1 ns for iron oxide; K and V are the anisotropy constant and volume of a nanoparticle respectively; k is the Boltzmann constant; T is the temperature. Since the bead signal is measured during relaxation where the interfering magnetizing field goes to zero, detection errors are significantly reduced.

The methods described in this disclosure leverages the short time constants and miniaturized components achievable in modern sub-micron CMOS technology. A fully integrated magnetic bead detector based on magnetic relaxation will eliminate external magnets, baseline calibration or reference sensors. Further, the CMOS bead relaxation detector can significantly reduce the power dissipation, detection time and system complexity while achieving high area-efficiency.

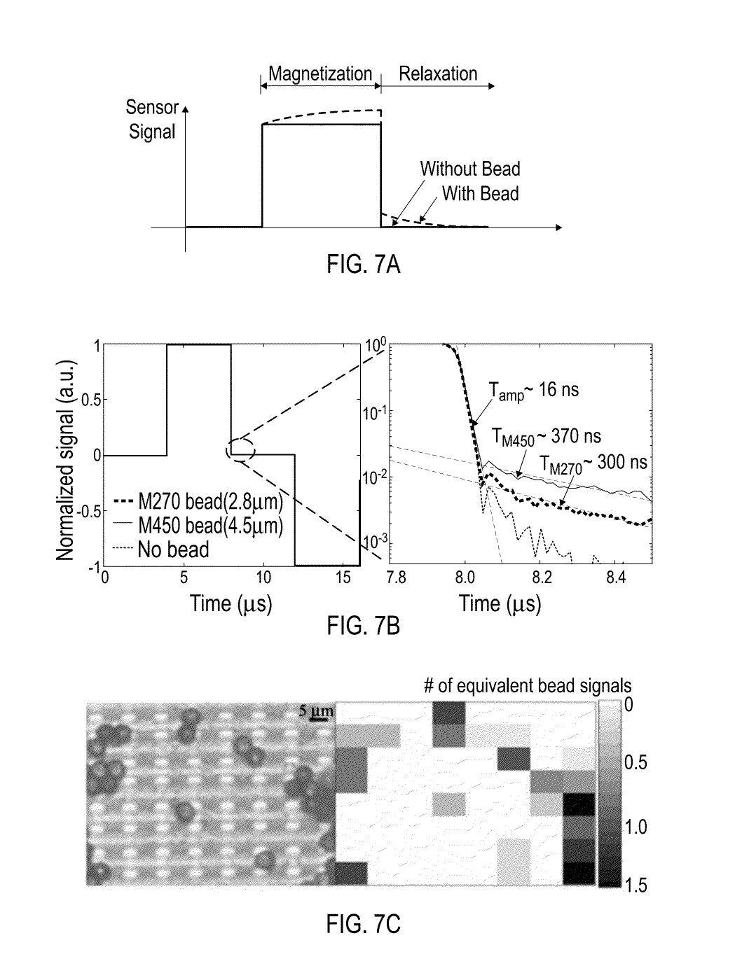

FIG. 7A illustrates an embodiment of the disclosed method and a conventional method for bead detection. In the conventional method, the bead signal is measured during magnetization and may be interfered by the magnetizing field which is usually several orders of magnitude larger. In contrast, in the relaxation detection method described in this disclosure, the bead signal is measured when the large magnetizing field is zero to ensure robustness. FIG. 7B illustrates a normalized signal of the measured relaxation of a single 4.5-μm and 2.8-μm bead. The zoom-in shows the relaxation traces in log-scale. FIG. 7C demonstrates the bead relaxation detected by a Hall sensor array. A 2-μl droplet of a diluted bead sample was added on the sensor surface and air dried (left). Due to the meniscus force, some beads were dragged to the sensor edge. The outputs from the 64-sensor array (right) matched well with the optical image. These results demonstrate the feasibility to use magnetic sensors as an imaging device for magnetic bead labels.

Analysis of multiple biomarkers on individual cells is beneficial for distinguishing target cells from contaminants (leukocytes, bead, debris, etc). It also provides insight into heterogeneity in cell populations. Conventional magnetic-based approaches are typically limited by one type of bead. To reliably and rapidly detect multiple magnetic labels, embodiments of this disclosure provide a method based on magnetic relaxation of multiple bead labels.

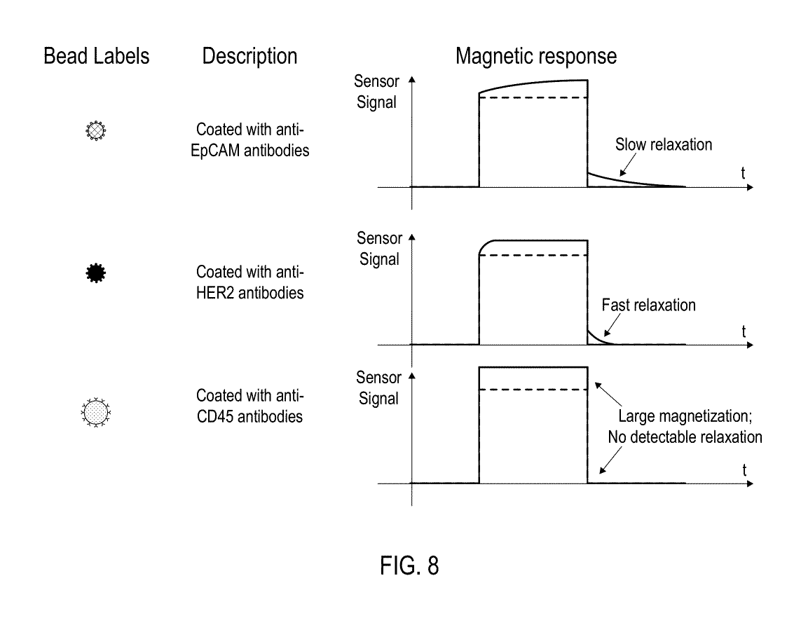

Therefore, in some embodiments, the target analyte in a sample may be labeled with beads having different relaxation characteristics. As shown in above equation (I), the Néel relaxation time constant depends strongly on the size and material of the nanoparticles inside a microbead. Numerous beads which show different relaxation characteristics are commercially available. By way of example, three types of beads may be chosen to label three surface markers, EpCAM, HER2, and CD45. The first type of bead may be coated with anti-EpCAM antibodies for surface marker EpCAM which is expressed by cancer cell line MDA-MB-453. The first type of bead may have a size about 5 μm and Néel relaxation time constant τ>10 μs. The second type of bead may be coated with anti-HER2 antibodies for surface marker HER2 which is also expressed by cancer cell line MDA-MB-453. The second type of bead may have a size about 5 μm and Néel relaxation time constant 100 ns<τ<1 μs. The third type of bead may be coated with anti-CD45 antibodies for surface marker CD45 which is expressed by normal leukocytes. The third type of bead may have a size about 8 μm and Néel relaxation time constant τ<100 ns. FIG. 8 illustrates the magnetic responses of the three types of beads with characteristic relaxation signatures. For example, the bead coated with anti-EpCAM antibody shows a slow magnetic relaxation whereas the bead coated with anti-HER2 antibody shows a fast magnetic relaxation. The bead coated with anti-CD45 antibody shows a large induced magnetization and no detectable relaxation.

The sensor array architecture shown in FIGS. 5A-5D allows each sensor element to detect only the local presence (“1”) or absence (“0”) of a bead, rather than measuring the signal from all the beads bound to a cell. Therefore, compared to bulk detection, this digital approach significantly relaxes the dynamic range requirement and is more immune to noise. Most cancer cells and contaminant can be distinguished by detector outputs and subsequently sorted.

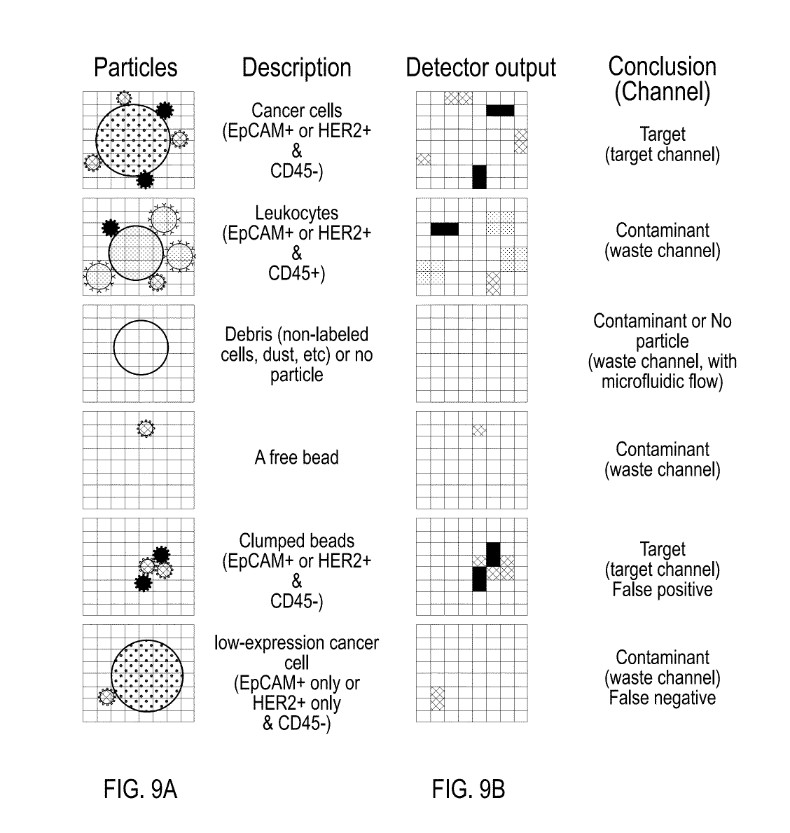

FIG. 9A is an optical image under microscope showing a target cancer cell and various contaminants on a detector array. Magnetic beads with different relaxation properties are coated with different antibodies. Cancer cells expressing EpCAM or HER2 are labeled with beads coated with anti-EpCAM and/or anti-HER2 antibodies. Leukocytes expressing CD45 are labeled with beads coated with anti-CD45 antibodies. Because of their large amount in the sample and their sticky surfaces, leukocytes may also be bounded by some beads coated with anti-EpCAM or anti-HER2 antibodies. Debris such as non-labeled cells, dust etc, free beads, clumped beads, and low-expression cancer cells are also shown.

FIG. 9B illustrates the detector output showing the detector responses to various particles. Individual particles are trapped to the surface of the Hall detectors where their magnetic responses are measured. For example, anti-EpCAM and anti-HER2 antibody coated beads labeled to a cancer cell are independently measured by the Hall sensors of the various sensing elements over which the beads are located. The measurement provides a characteristic detector output as shown. Likewise, anti-CD45 antibody coated beads labeled to a leukocyte cell are independently measured, which provides a characteristic detector output as shown. Debris, free beads, clumped beads, and low-expression cancer cells are also measured and their detector output is shown. The measured particle is then released and sorted to either a target channel or waste channel as will be described in greater detail below.

The detection errors in the disclosed method are rare and only cause negligible effect in sample purity. Detection errors and thus incorrect sorting decisions may be caused by ultra rare events such as clumped beads and low-expression cancer cell. Contaminating beads are in a small number as shown in FIG. 3, since most beads are filtered during sample preparation. Furthermore, the contaminating beads are manipulated and detected with low magnetic field (<10 mTesla) and thus are much less likely to agglomerate than in strong-field applications. More importantly, clumped beads sorted to the target channel do not affect sample purity since they do not bind to any cells. Cancer cells labeled with just one bead are also extremely rare subpopulation and may cause a yield loss less than 1%. When necessary, the detection errors due to these ultra rare events can be eliminated by morphological analysis with a light microscope focusing on the Hall detector.

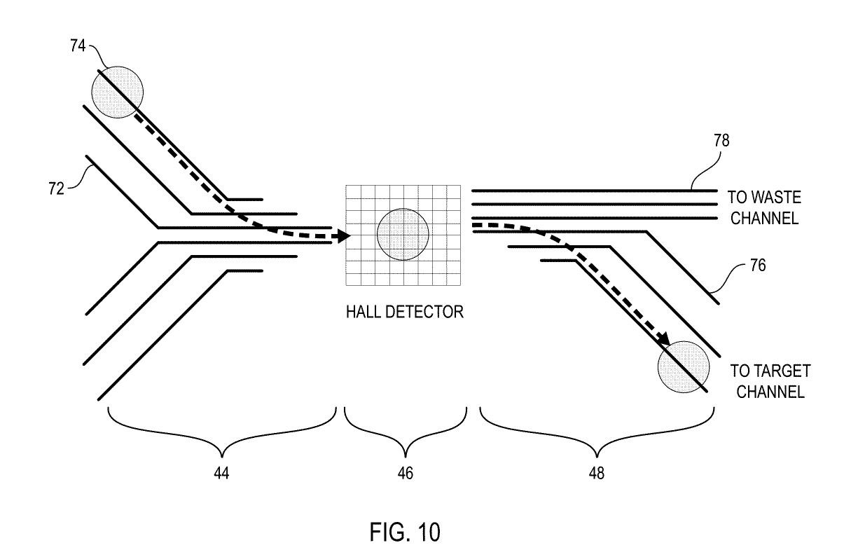

FIG. 10 schematically shows magnetic focusing and sorting according to some embodiments of this disclosure. As shown in FIG. 10, the focusing device 44 may include a wire structure 72 fabricated in the chip for guiding magnetic bead-labeled cells 74 to the detection device 46 through magnetic manipulation. The sorting device 48 may include a wire structure 76, 78 for directing magnetic bead-labeled cells passing through the detection device 48 to designated target channel or waste channel. The wire structure 72, 76, 78 may include one or more metal layers embedded in the chip, which are enabled by standard CMOS fabrication process. The current through each metal wire can be controlled by an on-chip logic circuit, and can be quickly turned on and off to generate localized magnetic field peaks. A superparamagnetic particle can be trapped or moved by the magnetic force exerted on it. By controlling the current patterns, the functions such as focusing, trapping, releasing, and sorting of a magnetic particle can be integrated with the Hall detector on a single CMOS chip.

Unlike conventional systems that use expensive, bulky and power-hungry external magnets to generate high-gradient field, embodiments of this disclosure use current conductors embedded in the chip, only microns away from chip surface, to locally generate all magnetic fields and gradient. This approach significantly reduces the system cost and saves power dissipation by orders of magnitude since its electromagnet is much closer to the bead. When implemented on a CMOS chip, local magnetic field patterns can be rapidly programmed by the integrated circuits to allow efficient manipulation of individual bead-bound cells with precise position control.

Cells labeled with magnetic beads can be suspended inside a microfluidic system. The chip may contain an array of microcoils which produces spatially-patterned microscopic magnetic fields on the surface of the chip. In a given magnetic field pattern, the bead-bound cells are attracted toward local field magnitude peak positions and become trapped there. Therefore, by reconfiguring the spatial field pattern and hence by moving the field magnitude peak positions, the individual bead-bound cells can be transported to their desired locations. The modification of the field pattern can be done by changing the current distribution in the microcoil array using integrated control electronics. For example, each microcoil may be connected to its own current source for independent magnetic field control. Using microscopic magnetic field patterns generated by a microcoil array circuit allows manipulation of individual cells, moving each cell along a different path.

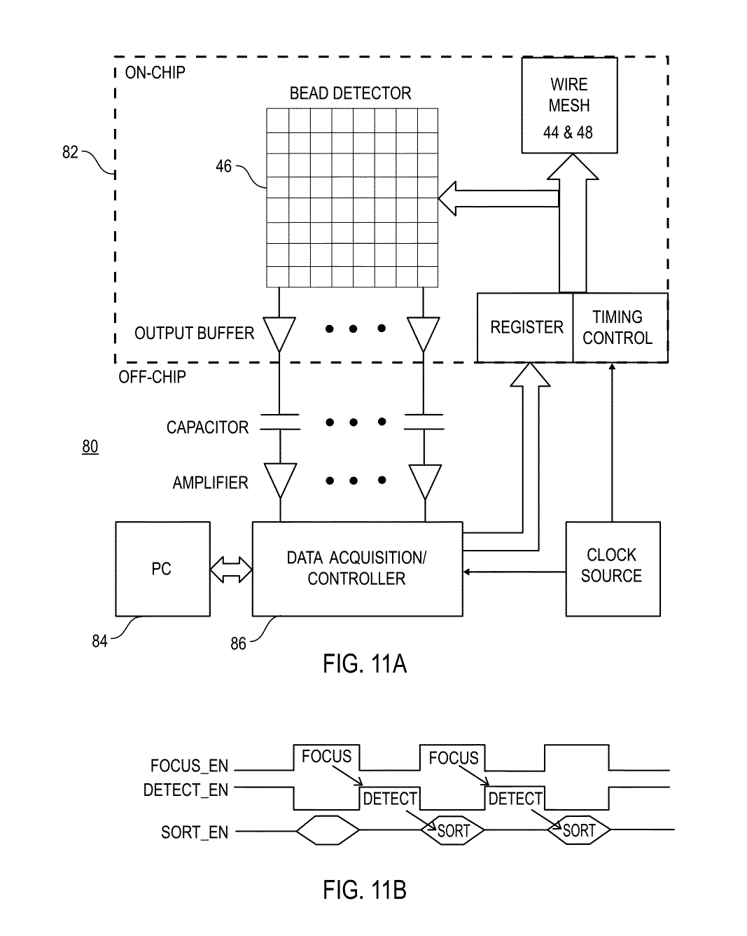

FIG. 11A is block diagram showing a system 80 including a magnetic flow cytometer chip 82 interfacing with a computer 84. The output of the Hall sensor array 40 can be amplified on board, digitized by a data acquisition device 86 and analyzed by the computer 84. The computer 84 may control the chip to perform its functions in a pipeline (FIG. 11B) so that the total isolation time can be determined by the step that takes the longest interval to process a cell. In a specific example, it took only 16 millisecond for a sensing element to detect a 4.5-μm bead with 16 dB signal-to-noise ratio (SNR), which can be translated into a probability of detection error <0.1%. It would take less than 1 second to read an array of 8×8 sensors in parallel with 20 dB SNR. However, sorting a bead-bound cancer cell to a microfluidic channel would take a longer time. It was demonstrated that a 20-μm diameter bovine capillary endothelial cell bound with magnetic beads can be moved by a force of 50 pN with an average speed of 6 μm/s on a silicon chip. In a scenario where a cancer cell is as large as 30 μm bound by only one 4.5-μm bead (e.g. due to low surface marker expression), it would take more than 100 mA current to move such a cell at a speed around 5 μm/s. Therefore it would take about 20 seconds to move this cell to a channel 100 μm away. However, the average sorting time for a cell would be much shorter, due to the facts that most cancer cells are bound with multiple beads, contaminants manipulation to the waste channel would also be assisted by the microfluidic flow, and leukocytes bound with larger beads and free floating beads can be manipulated at a much higher speed. It would take about 3 hour for a single magnetic flow cytometer to isolate up to 1000 cells. The sorting time can be significantly reduced by using multiple devices to process the sample in parallel, which is a proved benefit of microfluidics and microelectronics.

Exemplary embodiments of a rare cancer cell analyzer and sorter and embodiments of detecting and sorting rear cancer cells are described. Those skilled in the art will appreciate that various modifications may be made within the spirit and scope of the disclosure. All these or other variations and modifications are contemplated by the inventors and within the scope of the disclosure.

1. An apparatus comprising:
a substrate;
a detection device in the substrate configured to detect target analytes labeled with magnetic beads;
a focusing device in the substrate configured to introduce the labeled target analytes serially to the detection device;
wherein the detection device comprises a plurality of magnetic sensing elements each comprising a Hall-effect sensor configured to detect a magnetic characteristic of a magnetic bead labeled to a target analyte.

2. The apparatus of claim 1 wherein the plurality of magnetic sensing elements are arranged in arrays of rows and columns.

3. The apparatus of claim 1 wherein each of the magnetic sensing elements has a size about same as a size of a magnetic bead labeled to a cell.

4. The apparatus of claim 1 wherein each of the magnetic sensing elements has a size about 5×5 micrometers.

5. The apparatus of claim 1 wherein each of the plurality of magnetic sensing elements comprises an N-well plate and a pair of metal wires each being disposed adjacent along a side of the N-well plate configured to generate a magnetic field for magnetizing a magnetic bead labeled to a cell.

6. The apparatus of claim 1 wherein each of the magnetic sensing elements is configured to detect a magnetic relaxation characteristic of a magnetized magnetic bead labeled to a cell.

7. The apparatus of claim 1 wherein each of the magnetic sensing elements is configured to detect Néel relaxation signature of a magnetized magnetic bead labeled to a cell.

8. The apparatus of claim 1 wherein the focusing device comprises one or more current conducting wires configured to generate a magnetic field for urging the labeled target analytes to the detection device.

9. The apparatus of claim 1 wherein the focusing device and the detection device are fabricated in a single CMOS chip.

10. The apparatus of claim 1 further comprising a controller configured to control operation of the detection device and the focusing device.

11. The apparatus of claim 10 further comprising a computer display configured to display output of the plurality of magnetic sensing elements.

12. The apparatus of claim 1 further comprising a sorting device in the substrate configured to isolate target analytes from contaminates passing through the detection device.

13. The apparatus of claim 12 wherein the sorting device, the focusing device, and the detection device are fabricated in a single CMOS chip.

14. The apparatus of claim 12 further comprising a controller configured to control operation of the sorting device, detection device, and the focusing device.

15. The apparatus of claim 12 wherein the sorting device comprises a plurality of current conducting wires configured to generate magnetic fields for directing target analytes and contaminates in designated paths.

16. An apparatus comprising:
a substrate;
a detection device in the substrate configured to detect target analytes labeled with magnetic beads;
a focusing device in the substrate configured to introduce the labeled target analytes serially to the detection device; and
a sorting device in the substrate configured to isolate the labeled target analytes passing through the detection device;
wherein the detection device comprises a plurality of magnetic sensing elements each comprising a Hall-effect sensor configured to detect a magnetic characteristic of a magnetic bead labeled to a target analyte.

17. The apparatus of claim 16 wherein the focusing device, the detection device, and the sorting device are fabricated in a single CMOS chip.

18. The apparatus of claim 17 further comprising a controller configured to control operations of the focusing device, the detection device, and the sorting device.

19. The apparatus of claim 18 wherein the controller is configured to receive output from the plurality of magnetic sensing elements and provide signals to the sorting device for isolating labeled target analytes based on the output.

20. The apparatus of claim 19 further comprising a computer display configured to display the output of the plurality of magnetic sensing elements.

21. The apparatus of claim 17 wherein each of the plurality of magnetic sensing elements comprises an N-well plate and a pair of metal wires each being disposed adjacent along a side of the N-well plate configured to generate a magnetic field for magnetizing a magnetic bead.
